(19) United States
(12) Patent Application Publication
(10) Pub. No.: US 2005/0221456 A1
(43) Pub. Date: Oct. 6, 2005

(54) Use of indanoyl amide to stimulate secondary metabolism in Taxus sp.

(75) Inventors: Venkatesh Srinivasan, Sunnyvale, CA (US); Jennifer Alford, Ithaca, NY (US); Harald Heckenmuller, Hamburg (DE); Braden Roach, Trumansburg, NY (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Phyton Biotech, Inc., Ithaca, NY (US)

(21) Appl. No.: 11/056,396

(22) Filed: Feb. 14, 2005

Related U.S. Application Data
(60) Provisional application No. 60/543,921, filed on Feb. 13, 2004.

(51) Int. Cl. C12P 17/02

(52) U.S. Cl. 435/123; 549/510

(57) ABSTRACT

The invention is directed to a method for the production of taxanes by culturing suspension cells of Taxus sp. in a nutrient medium that comprises an indanoyl amino acid. The indanoyl amino acid may be added in batch mode or in a feed stream at any time of the culturing. In particular, synthetic compounds 6-Ethyl-indanoyl-isoleucine, 6-Bromoindanoyl isoleucine and 1-oxo-indane-carboxy-(L)-Isoleucine-methyl ester amide (1-OII) have been found to increase taxane production from Taxus cell cultures.

### CROSS-REFERENCE TO RELATED APPLICATION

This application claims the benefit of U.S. Provisional Application No. 60/543,921, filed Feb. 13, 2004, the disclosure of which is herein incorporated by reference in its entirety.

### FIELD OF THE INVENTION

The invention relates to the use of indanoyl amides and indanoyl amino acid conjugates such as coronolone, as well as methods for accumulating taxanes in plant cell cultures of taxane-producing species useful in the production of pharmaceutical compositions and therapeutic methods.

### REVIEW OF RELATED ART

Paclitaxel and analogs such as docetaxel are at the forefront of developments in cancer therapy, and there is increasing worldwide demand for these products. Since total chemical synthesis of paclitaxel is complicated and uneconomical, paclitaxel and its analogs are currently commercially produced through semisynthetic or biosynthetic methods. Semi-synthesis requires the use of advanced, naturally-occurring taxane precursors (closely related to the paclitaxel structure) and involves complex chemistry to complete the structural transformation of these precursors to paclitaxel or related taxanes. These precursors include 10-deacetylbaccatin III, baccatin III, N-acylated taxanes, etc. Various patents and articles disclose synthetic routes for diterpenoid cores, biosynthetic genes, enzymes or chemical conversion to desired taxanes. For examples of such disclosures, see e.g. U.S. Pat. No. 5,994,114; U.S. Pat. No. 5,200,534; U.S. Pat. No. 6,437,154; U.S. Pat. No. 6,287,835; U.S. Pat. No. 6,265,639; “Synthetic Routes to the Diterpenoid Cores of Phorbol and Resiniferatoxin,” Eckelbarger, J. D. 2001; “Taxol biosynthetic genes,” Walker, K. et al., Phytochemistry 58 (2001) 1-7.

Biosynthesis of paclitaxel is accomplished through the use of plant cell cultures of Taxus species. An advantage of a biosynthetic route is that a target compound or composition can be directly produced in a relatively pure form without the need for additional chemistry. Several publications disclose methods of producing taxanes by biosynthetic methods. U.S. Pat. No. 5,019,504 (Christen et al. 1991) describes the production and recovery of paclitaxel and taxane-like compounds by cell cultures of Taxus brevifolia. WO 93/171121 (Bringi et al., 1993) and U.S. Pat. No. 5,407,816 (Bringi et al., 1995) describe enhanced production and recovery of paclitaxel and other taxanes by cell cultures of Taxus species. Bringi et al. provide the earliest disclosure of an economically viable plant cell culture process for taxane production.

U.S. Pat. No. 6,248,572 (Choi et al., 2001) describes methods for producing paclitaxel by adding sugar, or AgNO₃ with sugar, into a culture medium in the course of semi-continuous culture of Taxus cells. Other related patents include, for instance, U.S. Pat. No. 5,665,576 and U.S. Pat. No. 6,428,989.

EP 0 683 232 A1 (Yukimune, 1995), EP 0 727 492 A2 (Yukimune et al.) and WO 97/44476 (Bringi et al., 1997) describe production of taxanes by culturing the tissue or cell in the presence of process enhancement agents, such as heavy metal compounds, heavy metal complex ions, heavy metal ions, amines or ethylene-resisting agents, and under controlled oxygen concentrations. These patent applications also disclose the beneficial effect of jasmonic acids and related compounds to enhance taxane yield in cell cultures of Taxus cells. The process enhancing compounds in these patent applications do not have aromatic rings.

Compounds related to jasmonic acids and their uses are described for instance in Haider, et al., “Structure-Activity Relationships of Synthetic Analogs of Jasmonic Acid and Coronatine on Induction of Benzo[c]phenanthridine Alkaloid Accumulation in Eschscholzia californica Cell Cultures,” Biol. Chem., Vol. 381, pp. 741-748, August 2000; Krumm, et al., “Leucine and Isoleucine Conjugates of 1-Oxo-2,3-dihydro-indene-4-carboxylic Acid: Mimics of Jasmonate Type Signals and the Phytotoxin Coronatine,” Molecules 1996, 1, 23-26; Miersch, et al., “Structure-activity Relations of Substituted, Deleted or Stereospecifically Altered Jasmonic Acid in Gene Expression of Barley Leaves,” Phytochemistry 50 (1999) 353-361; Schuler, et al., “Coronalon: a Powerful Tool in Plant Stress Physiology”; Yamada, et al., “Process for Production of Oxime Derivatives,” EP 86102811.6, Publication No. 0194554B1; Boudier, et al., “Sereoselective Preparation and Reactions of Configurationally Defined Dialkylzinc Compounds,” Chem. Eur. J. 2000, Vol. 6, No. 15, 2748-2761.

Haider et al., teaches that analogs of jasmonic acid, which elicit mechanotransduction, often do not also elicit the production of secondary metabolites. Moreover, Haider et al., Biol. Chem., Vol. 381, pp. 741-748, August 2000 (p. 744, col. 2, par. 1) also reported that the aromatization of coronatine rendered it planar and biologically ineffective. Haider, et al. point out that:
activation of defense genes and activation of genes for mechanotransduction appear to be regulated by different compounds that lie along the jasmonic acid biosynthetic pathway. Coronatine, the close structural analog of 12-oxo-phytodienoic acid, and the octadecanoids are strong inducers of tendril coiling. On the other hand, methyl jasmonate is much more effective in activating benzo[c]phenanthridine alkaloid accumulation than are the octadecanoids or coronatine. These results add support to the hypothesis that the various physiological responses, such as to herbivory or pathogens, and mechanotransduction among others, that are activated by the octadecanoid pathway can be separated by chemical derivatization of the inducer molecule (Blechert et al., 1997). This is strongly demonstrated by the different results obtained for defense gene activation herein with, for example, [three analogs of jasmonate] (all strong inducers of benzo[c]phenanthridine alkaloid accumulation in E. californica) and the failure of those same compounds to induce mechanotransduction in Bryonia by Blechert et al. (1999) close analogs.

Boland et al. describe aspects related to the physiologies of plants which have been exposed to jasmonic acids and/or coronatines. See for instance, Boland, et al., “Jasmonic acid and Coronatine Induce Odor production in plants”, Angewandte Chemie-International Edition, 43:1600-1602 (1995); Krumm, et al., “Induction of volatile biosynthesis in the Lima bean (Phaseolus lunatus) by lucine- and isoleucine conjugates of 1-oxo- and 1-hydroxyindan-4-carboxylic acid: Evidence for amino acid conjugates of jasmonic acid as intermediates in the octadecanoid signaling pathway”FEBS Lett. 377:523-529 (1995); and Schuler, et al., “Synthesis of 6-azido-1-oxo-indan-4-oyl isoleucine; a photoaffinity approach to plant signaling”, Tetrahedron, 55:3897-3904 (1999), Lauchli, et al., “Indanoyl Amino Acid Conjugates: Tunable Elicitors of Plant Secondary Metabolism,” The Chemical Record, Vol. 2, 000-000 (2002); and Schuler, et al., “6-Substituted Indanoyl Amino Acid Conjugates as Mimics to the Biological Activity of Coronatine,” WO 02/055480 A3.

WO 02/55480 describes 6-substituted indanoyl amino acids as plant regulators. Schuler, et al. (WO 02/55480) point out that “6-ethyl-1-oxo-indanoyl isoleucine methyl ester is a potent elicitor of the coiling reaction of the touch-sensitive tendrils of Bryonia diocia”. However, WO 02/55480 did not disclose cell culture nor use with Taxus sp. to improve taxane production.

Therefore, in view of the teaching of patent EP 727492 (Yukimune, et al.), in which coronatine was shown to induce paclitaxel and taxanes biosynthesis, it would be expected that a planar, aromatized analog of coronatine would not be biologically effective for this purpose.

### SUMMARY OF THE INVENTION

Significantly, none of these plant cell culture-based processes disclose the use of, for example, 6-ethyl-indanoyl-isoleucine (6-EII) for the use of improving or altering taxane productions of suspension culture cells, more particularly cells of the Taxus species. In view of the observation of Schuler et al. (WO 02/55480) that the indanoyl amino acid conjugate was a potent elicitor of tendril coiling (mechanotransduction), it is surprising that these specific analogs would elicit the production of taxane secondary metabolites from Taxus cell cultures. Furthermore, Haider, et al., indicate that aromatization, which renders coronatine analogs planar also renders them biologically inactive. However, 6-EII (which contains an aromatic ring) is both planar and active. Thus it is doubly surprising that the planar, coronatine-analog indanoyl amino acid conjugates, would be effective elicitors of taxane production in Taxus cell cultures.

One embodiment of the invention is directed to a method for the production of taxanes by culturing suspension cells of Taxus sp. in a nutrient medium comprising an indanoyl amino acid. Another embodiment of the invention is directed to a plant cell culture nutrient medium comprising indanoyl amino acids. The indanoyl compound may be added in a fed batch feed stream at any time during the culturing. In a preferred embodiment the nutrient medium in the suspension culture is partially depleted of its carbon source before a supplemental carbon source is included in the feed stream.

In each embodiment of the invention, multiple enhancement agents and/or inhibitors may be added to the nutrient medium. In a preferred embodiment the indanoyl amino acid is added in an amount effective to alter the profile of taxanes produced by the culture compared to the absence thereof. In another embodiment, the indanoyl amino acid is supplied in an amount effective to selectively increase baccatin III compared to the absence thereof.

For each of the recited embodiments, the indanoyl amino acid is selected from compounds described in WO 02/55480. More preferably the indanoyl amino acid is the unsubstituted indanoyl amino acid (the 1-oxo form) or a 6-substituted indanoyl isoleucine and derivatives thereof. Most preferably, the indanoyl amino acid is selected from 6-ethyl indanoyl isoleucine (6-EII), 6-Bromoindanoyl isoleucine (6-BII), 1-oxo-indane-carboxy-(L)-isoleucine-methyl ester amide (1-OII) or mixtures thereof. It should be appreciated that any one of the indanoyl amino acid compounds may be used alone to elicit the production of the desired taxanes.

For each of the recited embodiments, a preferred method of increasing taxane production includes the use of additional enhancement agents, more preferably selected from silver compounds and complexes, methyl jasmonate-related compounds, and phenylpropanoid inhibitors. Likewise, the production of preferred taxane products may be stimulated by supplying different biosynthetic precursors of those taxanes to the media at appropriate stages. Additionally, it is recognized in the art that rapid biomass growth and rapid production of product may be separated and different formulations of nutrient media and different culture conditions may be used for the growth and production phases.

In another embodiment the nutrient medium comprises an auxin, a compound with auxin-like growth-regulator activity or mixtures thereof. In another embodiment the nutrient medium comprises a silver ion, a silver compound, a silver complex, or mixtures thereof. In another embodiment, the nutrient medium comprises an inhibitor of phenylpropanoid metabolism. In a preferred embodiment, the inhibitor of phenylpropanoid metabolism is a compound with a methylene-dioxy group, more preferably the inhibitor of phenylpropanoid metabolism is MDCA (3,4-methylenedioxycinnamic acid) or related compounds such as methylenedioxynitrocinnamic acid, 3,4-methylenedioxyphenylacetic acid, methylenedioxyphenylpropionic acid, etc. In another embodiment, the nutrient medium further comprises an amino acid, more preferably the amino acid is glutamine.

In a preferred embodiment, the indanoyl amino acid is supplied in a protective amount with respect to silver toxicity.

### DETAILED DESCRIPTION OF THE INVENTION

The invention provides methods for increasing the accumulation of taxanes in plant cell cultures of taxane-producing species and methods for isolating and purifying such. The invention is directed to particular enhancement agents (indanoyl amides) used alone or in combination with other enhancement agents to improve yield of taxanes as paclitaxel, baccatin III and other taxane analogs.

Each of the documents recited throughout the specification is hereby incorporated by reference in its entirety to the extent not inconsistent with the disclosure herein. In particular, these references provide preferred methods to develop from callus culture both growth and production media which result in high yield of taxanes, including addition and manipulation of culture conditions to improve yield.

The taxanes are members of a family of compounds also known as the taxoids or taxane diterpenoids. The taxanes are characterized by the tricyclic diterpene taxane ring system. There are currently over 300 known taxanes. The terms “paclitaxel-like compounds,” or “taxanes,” are used interchangeably to describe a diterpenoid compound with a taxane ring. The taxanes may themselves possess antineoplastic activity or may be capable of modification to yield bioactive compounds. Various specific taxanes are also listed in WO 97/44476 (Bringi et al., 1997), EP 0 683 232 A1 (Yukimune, 1995) and EP 0 727 492 A2 (Yukimune et al.).

The term “taxane-producing cells” refers to any cells that are capable of producing taxane molecules under at least one set of culture conditions. The term “taxane-producing species” refers to any species that is capable of producing taxane molecules under at least one set of culture conditions. The term “taxane-producing cell culture” refers to any culture that contains “taxane-producing cells”. The biosynthetic tissue (i.e., the taxane-producing cells) may be selected from any taxane yielding species (or combinations thereof), as would be known to a person of skill in the art. Preferably, the Taxus species from which the tissue is selected is Taxus brevifolia, Taxus canadensis, Taxus cuspidata, Taxus baccata, Taxus globosa, Taxus floridana, Taxus wallichiana, Taxus media, Taxus chinensis and Taxus gena. More preferably, the Taxus tissue is selected from T. chinensis. Alternatively, the tissue may be selected from one or more Torreya species. The Torreya species is preferably, Torreya gradifolia or Torreya californica. The tissue may also be selected from one or more Corylus species. Preferably, the Corylus species is Corylus avellana. The use of combinations of cells from different species, variants, strains and/or genus, in any manner, is contemplated by the invention. For example, one or any combination of cells of Taxus species may be combined with one or any combination of Torreya species, without intending to be limited thereto. The use of tissue from plants that are hybrids, genetically altered and the like, are also contemplated.

The term “callus” is used to describe a mass of cultured plant cells that is structurally undifferentiated, and is cultivated on solidified medium. The term “suspension culture” is used to describe structurally undifferentiated cells that are dispersed in a liquid nutrient medium. It is understood that suspension cultures comprise cells in various stages of aggregation. A range of aggregate sizes are encountered in the suspensions with sizes ranging from tens of microns in diameter (single cells or few-aggregated cells) to aggregates many millimeters in diameter, consisting of many thousands of cells.

The term “nutrient medium” is used to describe a medium that is suitable for the cultivation of plant cell callus and suspension cultures. The term “nutrient medium” is general and encompasses both “growth medium” and “production medium.” The term “growth medium” is used to describe a nutrient medium that favors rapid growth of cultured cells. The term “production medium” refers to a nutrient medium that favors paclitaxel, baccatin III or total-taxane biosynthesis in cultured cells. It is understood that growth can occur in a production medium, production can take place in a growth medium and both growth and production can take place in a single nutrient medium. Preferably growth and production phases of taxane-producing cell culture are distinguishable, with independently optimized nutrient media.

When all the nutrients are supplied initially, and the culture contents comprising cells and product are harvested at the end of the culture period, the operating mode is termed a “single-stage batch process.” When a batch process is divided into two sequential phases, a growth and a production phase, with the medium being changed in between the two phases, the operating mode is termed a “two-stage batch process.” Within the contemplation of this invention, the transition from the growth medium through production medium may occur by an abrupt stepwise change, or progressively by a series of steps, or by progressive, continuous change. In one extreme, the progressive change is accomplished by progressive replacement of media of incrementally changing composition. In another alternative, the progressive change is accomplished by feeding one or more components of the production medium into the growth phase culture. This is one example of the fed-batch process. In a “fed-batch” operation, particular medium components such as nutrients and/or one or more enhancement agents are supplied either periodically or continuously during all or part of the course of a one-stage or a two-stage culture. A description of nutrients and enhancement agents may be found, for instance, in Table A or Tables 1 and 2 of WO 97/44476 (Bringi et al., 1997). Additionally a combination of abrupt and progressive changes may also be employed. In one example, some portion of the nutrient medium may be changed abruptly while other components are slowly fed.

In accordance with an embodiment of the invention, when a substantial portion, but not all, of the contents of a batch culture is harvested and fresh medium for continued cell growth and production is added, the process resembles a “repeated draw and fill” operation and is termed a “semi-continuous process.”

When fresh medium is continuously supplied, and effluent medium is continuously or repetitively removed, the process is termed “continuous,” in accordance with an embodiment of the present invention. According to another implementation, if cells are retained within the reactor, the process is termed a “perfusion mode.” If cells are continuously removed with the effluent medium, the continuous process is termed a “chemostat,” in accordance with another implementation of the invention.

Indanoyl Amides

Indanoyl amides contemplated by this invention as particularly useful enhancement agents for improving or altering taxane production are described below. Compounds and methods of making the compounds are described, for instance, in WO 02/55480, which has been incorporated by reference.

Coronatine has the following formula:

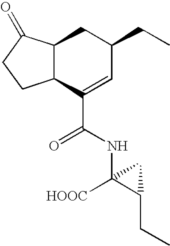

As described by Boland, et al., indanoyl amides were conceived as analogs of coronatine. Of the compounds described in WO 02/55480, preferably the compound is an indanoyl amino acid conjugate (also referred to as an indanoyl amino acid). Preferably the invention contemplates using compounds which may be represented, for instance, by the general formula of Boland et al. (WO 02/55480) reproduced below: 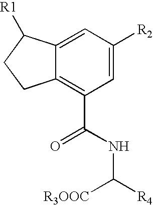
While Boland describes various substituents for the R groups, each of which are contemplated for use with the present invention, preferred R groups include:
R₁=a double bonded oxygen;
R₂=a lower alkyl group, such as a methyl group or an ethyl group, or a halogen which is preferably bromide;
R₃=methyl or hydrogen; and
R₄=the side chain of an amino acid. Preferably, the amino acid is a non-polar natural or synthetic amino acid. More preferably the amino acid is selected from glycine, valine, alanine, leucine, isoleucine and proline. In a particularly preferred embodiment, the amino acid is isoleucine.

According to this invention, indanoyl amides are used in place of or in addition to elicitors as described below. Preferably the indanoyl derivatives and analogs are used in the range between 10-500 micromol/l, more preferably between 50-250 micromol/l.

Cell Culture Methods and Media: Manipulation of Cultivation Conditions and Enhancement Agents

Cultivation conditions may be manipulated to favor production of desired taxanes. For example, reaction conditions such as temperature, pH, darkness, removal or addition of nutrient or other agent, change in concentration of nutrient or agent or combinations thereof may be manipulated.

The compositions may further comprise any of a number of additional components, such as nutrients and enhancement agents. Preferably, the composition comprises enhancement agents such as elicitors, jasmonate-related compounds, compounds affecting ethylene biosynthesis or action, especially inhibitors, inhibitors of phenylpropanoid metabolism, antisenescence agents, precursors and auxin-related growth regulators as discussed below.

Prior to initiation of the cell culture, the tissue may be selected based on other parameters, such as ability to favor formation of a particular taxane or taxane precursor under certain conditions, or treated (e.g., chemically, genetically or otherwise) to favor formation.

Production of secondary metabolites is a complex process, requiring coordinated action of many different enzymes to produce and sequentially modify precursors that are ultimately converted into target secondary metabolites. At the same time, secondary metabolite production will be lowered if other enzymes metabolize precursors of the desired metabolite, draining the precursor pools needed to build the secondary metabolites.

Taxanes are secondary metabolites that are produced through a series of many enzymatic steps, and several classes of enhancement agents are known to improve taxane biosynthesis. (See, for instance, Table A, and WO 97/44476 (Bringi et al., 1997), EP 0 683 232 A1 (Yukimune, 1995) and EP 0 727 492 A2 (Yukimune et al.)). Addition of one of these enhancement agents to a culture of taxane-producing cells will enhance the rate of taxane production. Furthermore, use of the enhancement agents will have at least some enhancing effect in many taxane-producing cultures, indicating that the overall production rate is determined not by a single rate-limiting step, but by a complex interaction between a plurality of limiting factors. Relief of any one of the limiting factors will enhance taxane production, although the magnitude of the enhancement will depend on particular culture conditions which determine the relative limiting effects of other steps in taxane biosynthesis, once a particular limitation has been relieved.

Culture conditions which affect the interaction between various limiting factors include the genetic make up of the cells, the composition of the culture medium and the gaseous environment, temperature, illumination and process protocol. The enhancement agent(s) added to a particular culture will usually be selected in view of the limiting factors in that culture, which may be determined empirically by comparing the effects of individual enhancement agents as set forth herein. Enhancement of taxane production may be achieved if more than one enhancement agent is present in the culture. Enhancement agent classes are: anti-browning agents, anti-senescence agents, anti-ethylene agents, plant growth regulators, precursors, inhibitors, elicitors, and stimulants. Representative enhancement agents and typical amounts requirements have been described for instance in the references cited in the background section, supra. In preferred embodiments, media may be specifically tailored to favor growth or taxane production. Based on the guidance provided herein and in view of the generally understood principles of plant cell physiology and metabolism, a person skilled in the art can readily independently develop media to favor growth or paclitaxel/taxane production.

Methods for callus formation and callus propagation of taxane-producing tissue are known in the art along with preferred means to vary growth morphology, productivity, product profiles, and other characteristics. Following establishment of the callus culture, cells are then cultured in production and/or growth media. In particular, production of taxanes in large quantities is facilitated by cultivating taxane-producing cells in suspension culture. Generally, suspension culture can be initiated using a culture medium that was successful in callus culture. However, the requirements for suspension culture, and particularly for highly efficient production of taxanes, may be better met by modification of the medium. It has been found that when taxane-producing cells are cultured in modified culture medium and processing parameters, the yield of one or more taxanes from the culture is substantially increased. Taxane-producing suspension cultures are capable of rapid growth rates and high cell densities when suitable nutrients and reaction conditions are used. It is a routine matter for those skilled in the art to incorporate, modify, and manipulate particular classes of components, and components from within a given class, to achieve optimum performance, based upon the guidance provided herein and in the patents which have been incorporated by reference in their entirety.

The invention contemplates the use of a biosynthetic process that is modified to yield the desired result. According to an embodiment of the invention, taxane-producing cells are cultivated in conditions under which taxanes can be produced. These include conditions favoring biomass accumulation and/or taxane production. The cultivation of taxane-producing cells is described in detail, for instance, in U.S. Pat. No. 5,407,816 (Bringi et al., 1995) and WO 97/44476, and modification of these taxane-producing conditions is described below and exemplified below in Examples 1 through 8. However, the invention is not intended to be limited thereto. Persons of skill in the art can prepare suitable taxane-producing cell cultures in accordance with the guidance provided herein in combination with references that have been incorporated by reference.

Various culture conditions and enhancements are known in the art, such as in the references described throughout this application; however preferred conditions are described below.

Preferred Cell Culture Embodiments

Cell culture initiation includes, for instance, surface sterilization of plant source material such as washing thoroughly with clean water, using a disinfectant such as hypochlorite, using wetting agents such as Tween or Triton, using antibiotics and optionally using antifungal agents. The plant part may then be used intact, or a portion of it may be used such as an embryo removed from a seed. The culture condition then uses typical nutrient media, temperature ranges and pH ranges suitable for Taxus callus formation known in the art. Similarly, gelling agents, reduction of pigmentation, activated charcoal, etc., and standard light/darkness cycles are used.

Callus propagation is the development of substantially undifferentiated cell mass attached to the plant part that is carefully removed and propagated as an undifferentiated culture. Culture conditions related to preferred media, pH ranges, preferred carbon sources, nitrogen sources, macro-salts and microsalts, vitamins and growth regulators are all described, for instance, in WO 97/44476. In addition to the procedures set out therein, preferred gelling agents include agar, hydrogels, gelatin and gelrite. Likewise, charcoal is preferably used for removing wastes and undesirable organic compounds. The inoculum is typically in the range of about 0.01-10 g/25 ml. Additionally, preferably subculturing techniques are utilized for the periodic serial transfer of portions of callus into a fresh source of nutrients.

Conditions for suspension culture are described, for instance, in WO 97/44476. In addition to the procedures set out therein, once initiated the suspension culture may be further cultivated either by separating the cells substantially from the medium (typically by filtration) and then reintroducing a portion to a medium containing nutrients or by transferring a volume of culture broth (cells and medium) into a medium containing nutrients or by allowing the cells to settle followed by removal of any portion of medium already present and reintroducing nutrient-containing medium. When cells are separated and transferred to a different nutrient-containing medium, the transferred amount may range from 0.3% to 30% on a fresh weight basis, although a fresh weight of 1%-25% would be preferred. Note that as the cells acclimate and/or grow, this fraction may change. When cells and media are transferred volumetrically, the ratio of the transferred volume to the final volume may be from 1% to substantially all of the volume. In this case, fresh nutrients may be supplied in a concentrated form so as to result in only a small volume increase. The culture may thus be divided into portions. Each portion may be used optionally for taxane production. The compositions of the nutrient containing media need not be the same for the different portions. Other ingredients not contained in the original culture medium may be added or items from the original medium may be omitted or altered in strength. The culture duration is typically at least 2 days. Additionally the duration of growth may be extended by supplementing a partially depleted medium with added nutrients.

All concentrations refer to average initial values in the extracellular medium. Concentrations in feed solutions and therefore, locally, concentrations in contact with the cells could be higher than that indicated. In addition to nutrients typically employed in plant cell culture, other ingredients may be included to aid taxane production. Ingredients especially suitable for taxane production include one or more selected from elicitors, stimulants, precursors, inhibitors, growth regulators, heavy metals, and/or ethylene inhibitory compounds. Elicitors include jasmonic acid and related compounds, tuberonic acid and related compounds, cucurbic acid and related compounds, coronatine and related compounds, 6-ethyl-indanoyl isoleucine and related compounds, 12-Oxo-phytodienoic acid and related compounds, systemin and related compounds, volicitin and related compounds, oligosaccharides, chitosan, chitin, glucans, cyclic polysaccharides, preparations containing cellular material from bacteria, fungi, yeasts, plants, insects, or material contained in insect saliva or secretions, etc., inhibitors of ethylene biosynthesis or action in plants, especially silver-containing compounds or complexes, cobalt, aminoethoxyvinylglycine, etc., inhibitors of phenylpropanoid metabolism such as compounds known to inhibit phenylalanine ammonia lyase, cinnamic acid hydroxylase, coumarate CoA ligase, methylenedioxycinnamic acid, methylenedioxynitrocinnamic acid, methylenedioxyphenylpropionic acid, in general, other compounds including a methylenedioxy functionality such as methylenedioxyphenylacetic acid, methylenedioxybenzoic acid, etc. Non-limiting examples of growth regulators and/or inhibitors and combinations thereof, without intending to be limited thereto, are included in Table A and the Tables disclosed in WO 97/44476 (Bringi et al., 1997). Amino acids include any common amino acid utilized in cell culture such as glutamine, glutamic acid, aspartic acid, α- or β-phenylalanine, etc., and the like.

Elicitors such as jasmonic acid-related compounds, etc., may be typically used at doses ranging from 0.01 micromoli to 1 mmol/l. Preferably this value will be between 1 and 500 micromol/l. Preparations containing cellular material may be added based on the concentration of a specific constituent of the preparation or as some fraction of the culture volume. Heavy metals and ethylene inhibitors such as silver salts or complexes, may be used at concentrations up to 1 mmol/l; however, typically the range will be 0.01 to 500 micromol/l. Other inhibitors may be used at concentrations of 1 micromol/l to 5 mmol/l. Aromatic compounds including a methylenedioxy functionality may be included at concentrations of 0.1 micromol/l to 5 mmol/l but more typically 1 micromol/l to 2 mmol/l. Growth regulators may be used at values ranging from 0.001 micromol/1 to 2 mmol/l but more typically they may be used at concentrations ranging from 0.01 micromol/l to 1 mmol/l. Precursors such as amino acids or terpenoid precursors may be used at concentrations ranging from 1 micromol/l to 20 mmol/l; however more typically, they will be used at concentrations ranging from 10 micromoli to 10 mmol/l. Following the guidance provided herein, it is possible for a person skilled in the art, through routine experimental approaches, to discover specific advantages of using materials singly or in combination outside the suggested ranges of usefulness. Such discoveries are considered to be within the scope of the present invention.

Ingredients provided to the cells may be provided in a number of different ways. Ingredients may be added in a particular stage of growth such as lag, exponential or stationary phases. All ingredients may be provided at once, and then after a suitable period of time, taxanes may be recovered. Alternatively not all ingredients may be provided all at once. Rather one or more of them may be provided at different times during the cultivation. Further, the additions may be discontinuous or staggered as to the time of initial contact and the duration of such provision may vary for different ingredients. Ingredients may be provided in a plurality of parts. Taxanes can then be recovered. One or more ingredients may be supplied as part of solutions separately contacted with the cell culture or portions thereof. Taxanes may be recovered from the entire culture or portions of culture (medium only, cells only or an amount of cells and medium together). Taxanes may be recovered any time during the cultivation or after the completion of the culture period. Portions of the culture may be removed at any time or periodically, and used either for taxane production and/or recovery or to further propagate the cells. Such cell-containing portions may be exposed further to nutrients or other ingredients as desired.

In one embodiment, medium containing nutrients or other ingredients may be added to replenish a portion or all of the removed volume. The replenishment (dilution) rate (volumetric rate of addition divided by the volume of liquid in the vessel) may vary between 0.1 times to 10 times the specific growth rate of the cells. Portions of such removed material may be added back into the original culture, for instance, cells and medium may be removed, the medium or cells may be used for taxane recovery and the remaining cells or medium may be returned. The supply rate of ingredients to the culture or levels of various ingredients in the culture may be controlled to advantageously produce and recover taxanes. Separate portions of the culture may be exposed to ingredients in any of the foregoing modes and then combined in proportions determined to be advantageous for taxane production. Also the cell-content of the culture may be adjusted to advantageously produce taxanes or propagate cells. Adjustment of cell-content may be advantageously combined with strategies for contacting with nutrients or other ingredients.

The levels of gases such as oxygen, carbon dioxide and ethylene may be controlled to advantageously produce one or more taxanes or to favor biomass accumulation. Routine cultivation in lab scale cultivation vessels held in an atmosphere of air, with typical closures such as sheets, plugs or caps result in dissolved oxygen levels below air saturation and levels of CO₂ and ethylene higher than that present in atmospheric air. Thus routinely, carbon dioxide levels in the head-space of the culture are typically greater than about 0.03%. However, the concentrations of carbon dioxide and/or ethylene may be adjusted to advantageously produce taxanes or propagate cells. Thus the inventors have discovered that for better taxane production, it is preferable that this level be higher than 0.1% (approximately 3-times atmospheric) to 10% and preferably between 0.3% and 7% CO₂. Ethylene may be preferably less than 100 ppm as measured in the gas phase in equilibrium with the liquid phase at that temperature and preferably less than 20 ppm. Dissolved gases can be controlled by using one or more methods comprising varying the agitation rate, the composition of aeration gas, the supply or venting rate of the aeration gas or by adjusting the total pressure in the cultivation vessel.

Gases may also be provided independently; for example, the sources of oxygen and CO₂ may be different. Agitation rates may be controlled between 1 and 500 per minute (rotations or oscillations of agitators or circulations of fluid). The supply rate of gas may be between 0.01 and 10 volumes or gas per volume of culture broth per minute and may be supplied directly into the culture liquid, or into a separate portion of liquid that is subsequently mixed with the rest of the culture, or into the head space of the culture. Typically, favorable dissolved oxygen concentrations for taxane production and Taxus cell growth may be controlled between 10% and 200% of air saturation at the operating temperature and preferably between 20% and 150% of air saturation. Of course, it is possible that for various operational reasons, e.g., temporary reduction in aeration, the dissolved oxygen level could be as low as zero for periods of time ranging from a few minutes to several hours. Specific useful combinations of oxygen, carbon dioxide, and ethylene, outside these ranges may be discovered through routine experimentation and are considered to be within the scope of this invention.

In one embodiment, media may be specifically tailored to favor growth or taxane production. Based on the guidance given above and based on generally understood principles of plant cell physiology and metabolism, a person skilled in the art would be able to design media to favor growth or paclitaxel/taxane production. For instance, one may, vary the temperature range (e.g., 0° C.-35° C., preferably 15° C.-35° C., more preferably 20° C.-30° C.). Likewise one may alter the periods of the light/dark cycle.

In a preferred embodiment, taxane producing cells are transferred from growth medium into production medium with a higher concentration of primary carbon source. The production medium also comprises a silver-containing compound or complex and methylene-dioxy-containing compound. Additionally, an indanoyl amino acid compound (e.g., 6-EII), glutamine and NAA (Naphthaleneacetic acid) are introduced into the culture 0-3 days after the cells and production medium are first contacted. Even more preferably, these ingredients are supplied in a feed stream. Supplemental glucose or maltose are preferably added as needed to the culture.

Methods for purification, recrystallization, isolation, extraction of taxanes from cell culture, plant tissues or mixtures comprising different taxanes are described, for instance, in U.S. Pat. No. 6,452,024; U.S. Pat. No. 6,215,000; U.S. Pat. No. 6,136,989; U.S. Pat. No. 6,124,482; U.S. Pat. No. 5,281,727; U.S. Pat. No. 5,380,916; U.S. Pat. No. 5,969,165; U.S. Pat. No. 5,900,367; U.S. Pat. No. 5,393,896; U.S. Pat. No. 5,393,895; U.S. Pat. No. 5,549,830; U.S. Pat. No. 5,654,448; U.S. Pat. No. 5,723,635; U.S. Pat. No. 5,736,366; U.S. Pat. No. 5,744,333; U.S. Pat. No. 5,756,098 and U.S. Pat. No. 6,008,385.

In using fed-batch, it has been found that cells can be sustained in a productive state for a prolonged period, and in fact, productivity of the cells could be enhanced. It will be apparent to the skilled artisan, that the composition of the feed may be varied to obtain the desired results such as extension of the production phase to increase taxane yield or extension of the growth phase to achieve higher biomass density. Selection of suitable conditions to achieve optimum productivity and performance is easily within the skill of the ordinary artisan in view of the teachings described herein. Similarly, variations of other operating parameters, such as the timing and duration of the addition and the rate of the addition of the fed-batch components, to achieve the desired results, are within the reach of the skilled artisan in view of the teachings described herein.

As documented, for instance, in Bringi WO 97/44476, the removal of spent medium and replenishment of fresh medium periodically, e.g., every few days, contributes to significant enhancement of total taxane and paclitaxel production, as well as to an increase in the amount of extracellular product.

The stimulatory effects of medium exchange may be due to removal of product in situ, which would prevent feedback inhibition and product degradation. Such positive effects of in situ product removal on secondary metabolite production and secretion in suspension cultures of unrelated plants have been documented by, among others, Robins and Rhodes (1986) and Asada and Shuler (1989). The periodic removal of spent medium incorporates the above advantages, and additionally, may serve to de-repress secondary biosynthesis by removing other, non-taxane inhibitory components (such as phenolic compounds) from the medium.

The replenishment of fresh medium to cells undergoing active biosynthesis may also enhance production by providing essential nutrients that have been depleted. For example, Miyasaka et al. (1986) were able to stimulate stationary phase cells of Salvia miltiorhiza to produce the diterpene metabolites, cryptotanshinone and ferruginol simply by adding sucrose to the medium. Presumably, biosynthesis had ceased due to carbon limitation in the stationary phase. The periodic-medium-exchange protocol used in the present invention may be beneficial as a result of any of the above factors. The replenishment of depleted medium components can also be accomplished through a feed stream (fed-batch) comprising the components.

It is contemplated that the amount of medium exchanged, the frequency of exchange, and the composition of the medium being replenished may be varied in accordance with various embodiments of the invention.

The ability to stimulate biosynthesis and secretion by medium exchange has important implications for the design and operation of an efficient commercial process in the continuous, semi-continuous or fed-batch mode.

Although any of the embodiments described above for producing taxanes in culture may be used alone, the techniques may also be used in combination with one another.

### EXAMPLES

The below examples are non-limiting examples. It will be apparent to one of ordinary skill in the art that many changes and modifications can be made thereto without departing from the spirit or scope of the invention as set forth herein.

### Example 1

### Cultivation of Taxus Cells

Taxus cell cultures are initiated from any suitable part of the Taxus plant using accepted techniques of plant cell culture. Substantially undifferentiated cells are propagated on solidified or liquid nutrient medium under the conditions described below except that cells cultivated on solidified medium do not require agitation. Taxus cells are cultivated at a temperature 22-28 degrees Celsius, at pH 4-7, in darkness, using agitation to mix the culture and providing oxygen and other gases and ventilation by contacting oxygen-containing gas with the cell suspension. The oxygen is maintained anywhere between 10% and 150% of air saturation at the operating temperature, and the carbon dioxide is maintained higher than 0.05%. The level of oxygen and other gases is controlled by means of adjusting the agitation, pressure, composition of gas, ventilation rate or feed rate of the gas. The medium contains components capable of supporting the growth of Taxus cells, for example sugar in the range of 1-100 g/l, a cumulative amount of nitrogen sources in the range of 1-100 mmol/l, and may include growth regulators such as auxin and/or cytokinin-like compounds, e.g., naphthalene acetic acid (NAA), phenoxyacetic acid and halogen substituted phenoxyacetic acids, picloram, dicamba, benzylaminopurine, kinetin, zeatin, thidiazuron, indole acetic acid, etc. The medium may optionally contain taxane substances such as baccatin III, or 10-deacetyl baccatin III, amino acids such as glutamine, alpha- or β-phenylalanine or others, methylenedioxycinnamic acid (MDCA), or methylenedioxynitrocinnamic acid or methylenedioxyphenylacetic acid or alpha-aminophenylacetic acid or related compounds, a source of silver ion for instance in the form of silver nitrate or silver thiosulfate or other ingredient capable of affecting ethylene biosynthesis or action. The inoculum concentration of Taxus cells may be in the range of 10 g fresh cell weight/i to 300 g fresh cell weight/I.

The medium also contains an indanoyl amide as described in the present application, optionally in combination with one or more jasmonic acid-related substance, and these materials may be added at the beginning of the culture, after the exponential growth phase, or intermittantly throughout the culture period. The other ingredients of the medium may be added all at one time or at different times during the cultivation and may be fed continuously or intermittently. The ingredients may be added either before or after the inclusion of plant cells in the culture broth. Further, nutrients or other ingredients may be additionally added during cultivation, if deemed useful. Optionally, the medium may be changed after a suitable period of cultivation whereby the cells are freshly exposed to a medium containing similar ingredients as described above. Such changes could involve changes to the amounts of sugars such as glucose, fructose, sucrose, maltose, etc., nitrogen sources such as nitrate, ammonium, or amino acids or casamino acids, etc. After a period of cultivation, the culture is harvested and the levels of taxanes are quantified using HPLC and specific taxanes are identified using LC/MS/MS. Taxanes can be recovered from these cultures by appropriate extraction and purification procedures.

For Experiments 2-8, Taxus cells were cultivated as callus cultures on solid medium and then further cultivated as suspensions of cell aggregates in liquid medium. Although taxanes can be produced by cultivation on solid medium, the use of liquid medium is preferred. The temperature of cultivation was controlled between 20 and 30 degrees Celsius.

### Example 2

### Fed Batch Addition of 6-EII

This experiment was carried out according to the following parameters. Results of 12-day fed batch treatment are described in Table 1 and demonstrate that 6-EII and methyl jasmonate have comparable activity. Taxus chinensis cells were cultivated as callus and then as suspended cells in growth medium (Medium A shown in Table B) containing 1% maltose and B5 salts according to the methods described above. After seven days of cultivation in this medium, the suspended cells were substantially separated from this medium and placed at about 20% (w/v) fresh cells into a production medium consisting of about 20% (v/v) spent growth medium and 80% (v/v) basal production medium (Medium B shown in Table B, but 1.25-fold concentrated) with an initial pH of 5.8 and containing 20 micromol/l MDCA and 50 micromol/l SLTS (Silver Thiosulfate). NAA (naphthaleneacetic acid), GLN (glutamine), and MJS (methyl jasmonate) or 6-EII (6-ethyl-indanoyl-isoleucine) were fed as part of a feed stream at the following rates: 1.66 micromol/l/d (NAA), 0.84 mmol/l/d (GLN), and MJS (2.51 micromol/l/d) or 6-EII (0.832, 1.665, 4.17, or 6.25 micromol/l/day). The gas phase was controlled to 25% oxygen and 4.5% CO₂ (balance nitrogen) and the temperature was held at 25±2 degrees Celsius. The cells were cultivated in the dark and were agitated at 100-150 rpm for the duration of the experiment. After 12 days of cultivation in production medium, the culture was harvested for taxanes.

The following results demonstrate that in cultures which are fed 6-EII (i.e. cumulative doses of 20-100 micromol/l) increments or methyl jasmonate (MJS), taxane production responds to 6-EII in a manner similar to methyl jasmonate.

TABLE 1
Effect of fed batch addition of 6-EII on taxane production

| | Cumulative 12-Day | | | |
|---|---|---|---|---|
| | Fed Batch Dose | Baccatin III | Paclitaxel | Total |
| Elicitor | (micromol/l) | mg/L | mg/L | mg/L |
| MJS | 30 | 273 | 490 | 1410 |
| 6-EII | 20 | 159 | 443 | 694 |
| 6-EII | 50 | 358 | 495 | 1301 |
| 6-EII | 75 | 605 | 556 | 1693 |
| 6-EII | 100 | 616 | 557 | 1702 |

### Example 3

### Batch Addition of 6-EII in Combination with other Medium Components

Taxus cells were cultivated using procedures similar to those used for Example 2. The media were composed of different combinations of 6-EII (or methyl jasmonate-MJS), NAA, SLTS, MDCA and Glutamine. All ingredients were added in a batch mode, i.e., all at once.

The data showed that 6-EII and MJS have comparable activity in improving taxane production. Thus 6-EII is no doubt effective when used by itself. However, the data also indicated several positive interactions between components of the media. The data showed that 6-EII works synergistically with silver to increase overall production of taxanes. In particular, the results demonstrate the ability of 6-EII to improve baccatin-III production without reducing the amount of paclitaxel produced. Also 6-EII and the auxin-type growth regulator NAA, interact positively to improve taxane production. The combination of 6-EII and auxin-type growth regulator is unexpectedly better than the use of the individual components. The data demonstrated that 6-EII and the amino acid glutamine interact favorably to improve taxane production. The data also surprisingly demonstrated 6-EII, silver (supplied as SLTS) and the auxin-type growth regulator, NAA, interacted to improve taxane production. Therefore the application of a combination of 6-EII with other enhancement agents such as NAA—an auxin-type growth regulator, SLTS—an example of silver containing compounds/complexes (inhibitors of ethylene action), methylenedioxy-containing compounds (inhibitors of phenylpropanoid metabolism) and glutamine (an example of an amino acid) along with the appropriately formulated medium (such as Medium B, Table B) results in improved taxane production.

### Example 4

### Effect of 6-EII in Combination with Other Medium Ingredients

This experiment was carried out according to the following parameters. Taxus chinensis cells were cultivated as callus and then as suspended cells on growth Medium A (Table B), according to the methods described above. After seven days of cultivation in this medium, the suspended cells were substantially separated from this medium and placed at approximately 20% (w/v) fresh cells into a basal production medium (Medium B, Table B) with an initial pH of 5.8. In addition to the components listed in Medium B, the medium additionally comprised 5 millimol/l glutamine. The gas phase was controlled to oxygen at 90-97% of the value in air and 2.5-6% CO₂; the temperature was held at 25±2 degrees Celsius. The cells were cultivated in the dark and were agitated at 150-200 rpm for the duration of the experiment. After 14 days of cultivation in production medium, the culture was harvested for taxanes.

Taxane yields in response to combinations of specific components of the medium were tested and compared to the yield obtained in the absence of a specific component or the absence of a specific combination of components. It was discovered that the inclusion of 6-EII in the range of 5-500 micromol/l, by itself resulted in an increase in taxane yield. However when combined with 20 micromol/l NAA (an auxin-type growth regulator), the yield improved by a significantly higher amount. The yield further increased when 50 micromol/l silver (an inhibitor of ethylene action, supplied as a thiosulfate complex) was added to the combination of 6-EII and NAA. And, when 20 micromol/l MDCA (3,4-methylenedioxycinnamic acid known to be an inhibitor of phenylpropanoid metabolism) was added to this combination, the yield increased even further. These surprising activities could not be predicted from the activities of the individual components. It is also possible to optimize the concentration of the components by titrating under particular conditions, such as using 50-200 micromol/l of indanoyl amino acid, such as 6-EII, in combination with 20 micromol/l NAA or with 20 micromoll NAA and 50 micromol/l SLTS or with 20 micromol/l NAA, 50 micromol/l SLTS and 20 micromol/l MDCA, to maximize the desired result.

### Example 5

### Effect of Silver Nitrate and 6-EII on Taxane Production

Taxus chinensis cells were cultivated as callus and then as suspended cells on growth medium (Table B, Medium A) according to the methods described above in Examples 1 and 2. After seven days of cultivation in this medium, the suspended cells were substantially separated from this medium and placed at ca. 20% (w/v) fresh cells into Medium B (Table B) at an initial pH of 5.8. In addition to the components listed in Table B, 20 micromol/l NAA, 20 micromol/l MDCA, 5 mmol/l GLN, MJS or 6-EII and silver as a nitrate salt or thiosulfate complex were added to the medium at various levels. The gas phase was controlled to oxygen at 90-97% of the levels in air and CO₂ at 2.5-6%; the temperature was held at 25±2 degrees Celsius. The cells were cultivated in the dark and were agitated at 150-200 rpm for the duration of the experiment. After 14 days of cultivation in production medium, the culture was harvested for taxanes.

The data demonstrated that 6-EII and silver act synergistically. Thus neither 6-EII or silver when used alone produce the magnitude of the effect observed when the cells were exposed to the combined presence of these agents. In this example, silver nitrate and silver thiosulfate are used as illustrative examples of silver containing compounds or complexes. The inventors have discovered that several other silver containing compounds or complexes may be used. Examples of other suitable silver complexes may be found for instance in the disclosures discussed in the background section such as WO97/44476 and U.S. Pat. No. 6,428,989.

### Example 6

### Effect of 6-Bromoindanoyl Isoleucine (6-BII) on Taxane Production

This experiment was carried out according to the following parameters. Taxus chinensis cells were cultivated as callus and then as suspended cells on growth medium (Medium A, Table B) according to the methods described above. After seven days of cultivation in this medium, the suspended cells were substantially separated from this medium and placed at approximately 20% (w/v) fresh cells into a basal production medium (Medium B, Table B) with an initial pH of 5.8. In addition to the ingredients listed in Table B, the medium comprised 20 micromol/l NAA, 20 micromol/l MDCA, 5 mmol/l GLN, and 50 micromol/l silver as a thiosulfate complex. 6-BII was added to the production medium at levels indicated in the Table 2 below. The gas phase was controlled to oxygen at 90-97% of the levels in air and 2.5-6% CO₂; the temperature was held at 25±2 degrees Celsius. The cells were cultivated in the dark and were agitated at 150-200 rpm for the duration of the experiment. After 14 days of cultivation in production medium, the culture was harvested for taxanes.

The data show that, like 6-EII, 6-BII (a halogen substituted derivative) was also an effective inducer of taxanes production by T. chinensis cell cultures. The data also exemplify determination of an optimum 6-BII level, which may likewise be determined by titrating other indanoyl amides in taxane-producing cell cultures.

TABLE 2
Effect of 6-Bromoindanoyl Isoleucine (6-BII) on taxane production

| Added | Dose | Baccatin | Paclitaxel | Total taxanes |
|---|---|---|---|---|
| compound | (micromol/l) | III (mg/l) | (mg/l) | (mg/l) |
| None | 0 | 66 | 229 | 364 |
| MJS | 45 | 154 | 574 | 912 |
| 6-BII | 5 | 92 | 235 | 423 |
| 6-BII | 20 | 114 | 230 | 441 |
| 6-BII | 50 | 174 | 254 | 580 |
| 6-BII | 100 | 313 | 360 | 962 |
| 6-BII | 200 | 307 | 375 | 1060 |
| 6-BII | 500 | 1 | 0 | 105 |

### Example 7

### Effect of 1-oxo-indane-carboxy-(L)-isoleucine-methyl Ester Amide (1-011) on Accumulation of Total Taxanes by Taxus Cell Suspension Culture

This experiment was carried out according to the following parameters. Taxus chinensis cells were cultivated as callus and then as suspended cells on growth medium (Medium A, Table B) according to the methods described above. After seven days of cultivation in this medium, the suspended cells were substantially separated from this medium and placed at ca. 20% (w/v) fresh cells into a basal production medium (Medium B, Table B) with an initial pH of 5.8. In addition to the ingredients listed in Table B, the medium also contained 20 micromol/l NAA, 20 micromol/l MDCA, 5 mmol/l GLN, and 50 micromol/l silver as a thiosulfate complex. 1-OII was added to the basal production medium at levels indicated in the table below. The gas phase was controlled to oxygen at 90-97% of the value in air and 2.5-6% CO₂; the temperature was held at 25±2 degrees Celsius. The cells were cultivated in the dark and were agitated at 150-200 rpm for the duration of the experiment. After 14 days of cultivation in production medium, the culture was harvested for taxanes.

The data show that like the 6-substituted derivatives, 6-EII and 6-BII discussed above, the 6-unsubstituted indanoyl isoleucine is also an effective inducer of taxanes production in Taxus suspension culture.

TABLE 3
Effect of 1-oxo-indane-carboxy-(L)-Isoleucine-methyl ester amide (1-OII)
on Accumulation of Total Taxanes by Taxus Cell Suspension Culture

| | Dose | Baccatin | | Total taxanes |
|---|---|---|---|---|
| Compound | (micromol/l) | III (mg/l) | Paclitaxel(mg/l) | (mg/l) |
| None | 0 | 49 | 170 | 277 |
| MJS | 45 | 226 | 432 | 854 |
| 1-OII | 5 | 50 | 192 | 300 |
| 1-OII | 20 | 72 | 248 | 400 |
| 1-OII | 50 | 112 | 349 | 580 |
| 1-OII | 100 | 242 | 409 | 822 |
| 1-OII | 200 | 375 | 407 | 1055 |
| 1-OII | 500 | 45 | 67 | 368 |

### Example 8

### Effect of Fed Batch Supply of 6-EII and the Effect of Combining with Supplementation of the Culture with Additional Sugar

Taxus chinensis cells were cultivated as callus and then as suspended cells in growth Medium A (Table B) according to the methods described above. After seven days of cultivation in this medium, the suspended cells were substantially separated from this medium and placed at ca. 20% (w/v) fresh cells into a 1.25-fold concentrated Medium B) (Table B) at an initial pH of 5.8, to which was added 20% (v/v) spent medium from the end of the culture in medium A. In addition 20 micromol/l MDCA, and 50 micromol/l silver as a thiosulfate complex were also added. NAA, GLN, and 6-EII were fed as part of a feed stream at the following rates: 1.66 micromol/l/d (NAA), 0.84 mmol/l/d (GLN), and 4 micromol/l/day (6-EII). The gas phase was controlled to 25% oxygen and 4.5% CO₂ (balance nitrogen) and the temperature was held at 25±2 degrees Celsius. The cells were cultivated in the dark and were agitated at 100-150 rpm for the duration of the experiment. After a period of cultivation (12 days in this case) under these conditions, the culture was partially depleted of primary carbon source. At this time the feed stream comprising NAA, GLN and 6-EII was supplemented with glucose at 400 g/l so that the cells were provided with adequate supply of carbon source and so that the duration of taxane production could be prolonged. The culturing was thus continued for an additional period of 16 days. The culture was harvested for taxanes at this time, and the amount of taxanes was quantified using established analytical procedures.

The results indicate that in contrast to a batch mode of cultivation in which the availability of medium components can limit the duration of the taxane production, supplying additional sugar in a fed batch mode permits the extension of this duration. Further, high taxane production can be accomplished by supplementation of sugar through small additions in a fed batch mode rather than in one abrupt, addition of the entire amount.

The foregoing describes preferred embodiments of the present invention along with a number of possible alternatives. These embodiments, however, are merely for example and the invention is not restricted thereto. Therefore the results of the Examples can be applied to Taxus species in general and are not limited to Taxus chinensis. In addition, the conditions of the Examples, such as the concentrations of the added components, are not limited.

TABLE A

(1-amino-2-phenylethyl)phosphonic Acid
(APEP)
(1-amino-2-phenylethyl)phosphonous Acid
(APEPi)
(3,5-Dimethoxy-4-hydroxy cinnamic Acid
(E)-2-aminomethyl-3-phenylpropenoic Acid
(S)-2-Aminooxy-3-phenylpropanoic Acid
(S)-4-Nitro-Phenylalanine
(S)-alpha-Aminooxy-β-phenylpropanoic
Acid
1-amino-3′,4′-dichlorobenzylphosphonic
Acid
1-amino-3-phenylpropylphosphonic Acid
1-Aminobenzotriazole
1-aminobenzylphosphonic Acid
2,3,4,5-Tetrafluorobenzoic Acid
2,3,5-Trichlorobenzoic Acid
2,3,5-Triiodobenzoic Acid
2,3,6-Trifluorobenzoic Acid
2,3-Dichlorobenzoic Acid
2,3-Difluorobenzoic Acid
2,4,6-Trichlorobenzoic Acid
2,4-Carbonyldibenzoic Acid
2,4-Dichlorophenoxyacetic acid
2,4-Dichlorobenzoic Acid
2,4-Difluorobenzoic Acid
2,4-Dimethoxybenzoic Acid
2,5-Dichlorobenzoic Acid
2,5-Difluorobenzoic Acid
2,5-Dimethoxybenzoic Acid
2,6-Dichlorobenzoic Acid
2,6-Dimethoxy-3-nitrobenzoic Acid
2,6-Dimethoxybenzoic Acid
2-Amino-2,5-Dichlorobenzoic Acid
2-aminoindan-2-phosphonic acid (AIP)
2-amino-indene-2-phosphonic acid
2-Bromo-5-methoxybenzoic Acid
2-Chlorobenzoic Acid
2-Fluoro-6-iodobenzoic Acid
2-Fluorobenzoic Acid
2-Fluoro-β-alanine
2-Hydroxy-4,6-dimethoxybenzoic Acid
2-hydroxyphenylaminosulphinyl acetic acid
1,1-dimethyl ester (OH-PAS)
2-Iodobenzoic Acid
2-Methoxycinnamic Acid
2-Naphthoic Acid
2-Nitrocinnamic Acid
3-(2-Hydroxyphenyl)propionic Acid
3-(3,4-Methylenedioxyphenyl)propionic
Acid
3-(3-Fluoro-4-methoxybenzoyl)propionic
Acid
3-(3-Methoxyphenyl)-beta-alanine
3-(4-Bromobenzoyl)propionic Acid
3-(4-Chlorobenzoyl)propionic Acid
3,4-(Methylenedioxy)-6-nitro-cinnamic
Acid
3,4-(Methylenedioxy)cinnamic Acid
3,4-Dichlorobenzoic Acid
3,4-Dimethoxy-6-nitrocinnamic Acid
3,4-Dimethoxyphenylacetic Acid
3,4-Methylenedioxybenzoic Acid
3,4-Methylenedioxyphenylacetic Acid
3,4-trans-Dimethoxycinnamic Acid
3,5-diaminobenzoic acid
3-nitrosobenzoic acid
4-nitrosobenzoic acid
nitrosobenzoic acids
3,5-Dibromobenzoic Acid
3,5-Dichloro-4-hydroxybenzoic Acid
3,5-Difluorobenzoic Acid
3,5-Dimethoxy-4-hydroxybenzyhydrazide
3,5-Dimethoxybenzoic Acid
3-Aminobenzamide
3-Benzoylpropionic Acid
3-Chloro-4-hydroxybenzoic Acid
3-Chlorobenzoic Acid
3-Fluorobenzoic Acid
3-Iodobenozic Acid
3-Nitrobenzoic Acid
3-Nitrocinnamic Acid
4-aminobenzoic acid
4-(Dimethylamino)cinnamic Acid
4-(Hydroxyphenyl)pyruvic Acid
4-Amino-DL-Phenylalanine
4-fluor-(1-amino-2-phenylethyl)phosphonic
acid
4-Fluoro-2-(trifluoromethyl)benzoic Acid
4-Fluorobenzoic Acid
Alternative benzoyl: CoA substrate
4-Fluorocinnamic Acid
4-Fluoro-D-Phenylalanine
4-Fluoro-L-Phenylalanine
4-Hydroxybenzoic Acid
4-Hydroxycinnamic Acid
4-Iodobenzoic Acid
4-Iodophenoxyacetic acid
4-Methoxybenzoic Acid
4-Methoxycinnamic Acid
4-Nitrocinnamaldehyde
4-Nitrocinnamic Acid
5-Fluoro-2-methylbenzoic Acid
5-Nitro-2-(3-phenylpropylamino)-benzoic
acid
6-Fluoro-2-hydroxybenzoic Acid
6-Methoxy-2-benzoxazoline
Ag-containing compounds or complexes
alpha-aminooxy acetic acid (AOA)
Ammonium Oxalate
Benzoic Acid
Benzoyl Chloride
Benzyl Cinnamate
beta-(2-hydroxy-3-methylphenyl)alanine
beta-(2-hydroxy-5-methylphenyl)alanine
beta-chloroethyltrimethylammonium
Beta-Phenyl-DL-serine
beta-phenylethylalanine
Both +/− -2-aminomethyl-3-phenylpropanoic
acid
Bromobenzoic Acid
Caffeic Acid
carbobenzyloxy-beta-alanine
Chlorogenic Acid
Cinnamic Acid
Cinnamonitrile
Cinnamylalcohol
Cinnamyledeneacetophenone
Cinnamylidene Malonic Acid
Co²⁺ (Cobalt) salts
Coumaric Acid
Cu²⁺ (Copper) salts
Diethyldithiocarbamic Acid (Na)
Dihydrocaffeic Acid
Dithiothreitol
DL-3,4-Dihydroxyphenylalanine
DL-3-aminobutyric acid
DL-3-Fluorophenylalanine
DL-aspartic acid
DL-o-Chlorophenylalanine
DL-p-Chlorophenylalanine
Ethyl Benzoate
Ethyl-3-nitrocinnamate
Ethyl-4-nitrocinnamic Acid
Ferulic Acid
Fumonisin
Gallic Acid
glycyl-beta-alanine
Glycyl-L-Phenylalanine
Glyphosate
Hg²⁺ (Mercury)-containing salts or
compounds
Hydroxylamine
Isonicotinate
Jasmonic acid and derivatives
Kaempferol
D(+)-1-amino-2-phenylethylphosphonic
Acid
L-3,4-Dihydroxyphenylalanine
L-alpha-aminooxy-beta-phenylpropionic
acid (AOPP)*
L-alpha-Aminooxyphenylpropanoic Acid
Lanthanum
L-beta-Homo-Phenylalanine
LiCl (Lithium Chloride)
L-Phenylalanine-2-Naphthylamide
L-Phenylalanine-p-Nitroanilide
L-tyrosine benzyl ester
Malonic Acid derivatives
MDCA (3,4-methylenedioxycinnamic acid)
Methyl Benzoate
Mg²⁺ (Magnesium)
m-Methylcinnamic Acid
Methyl jasmonate
N-(2-hydroxy-3-methoxy-5-
nitrobenzylidene)beta-alanine
N-(2-Hydroxyethyl)-beta-alanine
N-(3,4,5,6-tetrahydro-2H-azepin-7-YL)-
beta-alanine
n-(4-chlorophenyl)-N-tosyl-beta-alanine
N-(gamma-L-Glutamyl)Phenylalanine
N,N-dicyclohexylcarbodiimide
N,N-Dimethyl-L-Phenylalanine
N-Acetyl-imidazole
n-acetyl-1-aspartic acid
N-Acetyl-L-Phenylalanine
N′-benzoyl-beta-alanine methyl ester
N-Cinnamylpiperazine
N-cyclohexyl-beta-alanine
Ni²⁺ (Nickel)-containing salts or compounds
nicotinate
Nitrocinnamoyl alcohol
N-Maleoyl-beta-alanine
N-propyl-4-penenoic acid (metabolite of
VPA (valproic acid))
O-Benzylhydroxylamine (OB-HA)
o-Chlorocinnamic Acid
o-Hydroxycinnamic Acid
o-Methylcinnamic Acid
o-phenanthroline
o-Sulfobenzoic Acid
p-Aminobenzoic Acid
p-Amino-L-Phenylalanine
p-Chloromercuribenzoic Acid
p-Coumaric Acid
Pentostatin
p-Fluoro-DL-Phenylalanine
p-Fluoro-D-phenylalanine
Phenylacetic Acid and analogs
Phenylhydrazine
Phenyllactic Acid
Phenylpropiolic Acid
Phenylpropionic Acid
Phenylpyruvic Acid
p-Hydroxybenzoic Acid
p-Hydroxymercuribenzoic Acid
picolinate
p-Iodo-Phenylalanine
p-mercuribenzoate
p-Sulfamoylbenzoic Acid
para-substituted benzoic acids
Salicylic Acid
Semicarbazide
Silver nitrate or other silver salts
Silver thiosulfate
Spermidine (Spd³⁺)
Spermine (Spm⁴⁺)
Sulfabenzamide
t-cinnamate
tetcyclacis
Thiobenzoic Acid
Trans-2,4-Dimethoxycinnamic Acid
Trans-3,4-Difluorocinnamic Acid
trans-Coumaric Acid
trans-methyl cinnamate
Vanillic Acid
Zn²⁺ (Zinc)

TABLE B
Compositions of Medium A and Medium B

| | Medium | |
|---|---|---|
| | Medium A | Medium B |
| Component | mg/L | mg/L |
| Ammonium Sulfate | 134 | 33.5 |
| Ascorbic acid | 100 | — |
| Aspartic acid | 1330 | — |
| Boric Acid | 3 | 0.75 |
| Calcium Chloride Anhydrous | 113.24 | 28.3 |
| Calcium Chloride Dihydrate | — | 50 |
| Cobalt Chloride Hexahydrate | 0.025 | 0.00625 |
| Cupric Chloride Dihydrate | — | 0.01 |
| Cupric Sulfate Pentahydrate | 0.025 | 0.00625 |
| Disodium EDTA Dihydrate | 37.3 | 9.31 |
| Ferrous Sulfate Heptahydrate | 27.8 | 6.96 |
| Folic Acid | — | 10 |
| Glutamine | 292 | — |
| Glycine | | 50 |
| Magnesium Sulfate Anhydrous | 122.09 | 30.52 |
| Maltose | 10,000 | 70,000 |
| Manganese Sulfate Monohydrate | 10 | 27.5 |
| Myo-Inositol | 100 | 25 |
| Nicotinic Acid (Free Acid) | 1 | 0.25 |
| Picloram | 2.415 | — |
| Potassium Iodide | 0.75 | 0.188 |
| Potassium Nitrate | 2500 | 625 |
| Pyridoxine hydrochloride | 1 | 10.25 |
| L-Serine | — | 50 |
| Sodium Molybdate Dihydrate | 0.25 | 0.0625 |
| Sodium Phosphate Monobasic Anhydrous | 130.5 | 32.63 |
| Thidiazuron | 0.022 | — |
| Thiamine hydrochloride | 1 | 2.5 |
| Zinc Sulfate Heptahydrate | 2 | 0.5 |

1. In a method for production of taxanes by culturing cells of Taxus sp. in suspension culture in a nutrient medium, an improvement wherein an indanoyl amino acid is added to the nutrient medium.

2. The method of claim 1, wherein the indanoyl amino acid is provided in a fed batch feed stream.

3. The method of claim 2, wherein the suspension culture medium is partially depleted of carbon source before supplemental carbon source is included in the feed stream.

4. The method of claim 1, wherein the indanoyl amino acid is added to the nutrient medium in an amount effective to alter the ratio among taxanes produced by the culture relative to the ratio produced in the absence of the indanoyl amino acid.

5. The method of claim 1, wherein the indanoyl amino acid is added to the nutrient medium in an amount effective to selectively increase baccatin III relative to the amount produced in the absence of the indanoyl amino acid.

6. The method of claim 1, wherein the indanoyl amino acid is a 6-substituted indanoyl amino acid.

7. The method of claim 1, wherein the indanoyl amino acid is selected from the group consisting of 6-ethyl indanoyl isoleucine (6-EII), 6-Bromoindanoyl isoleucine (6-BII), and 1-oxo-indane-carboxy-(L)-isoleucine-methyl ester amide (1-OII).

8. The method of claim 1, wherein the nutrient medium comprises at least one enhancement agent and/or inhibitor.

9. The method of claim 1, wherein the nutrient medium comprises an auxin, a compound with auxin-like growth-regulator activity or both.

10. The method of claim 1, wherein the nutrient medium comprises a silver ion, a silver compound, a silver complex, or mixtures thereof.

11. The method of claim 1, wherein the indanoyl amino acid is supplied in a protective amount with respect to silver toxicity.

12. The method according to claim 1, wherein the nutrient medium comprises an inhibitor of phenylpropanoid metabolism.

13. The method according to claim 12, wherein the inhibitor is a compound with a methylene-dioxy group.

14. The method according to claim 13, wherein the compound is 3,4-methylenedioxycinnamic acid, methylenedioxynitrocinnamic acid, methylenedioxyphenylpropionic acid, or 3,4-methylenedioxyphenylacetic acid.

15. The method according to claim 1, wherein the nutrient medium further comprises an amino acid.

16. The method according to claim 15, wherein the amino acid is glutamine.

17. A plant cell culture nutrient medium comprising indanoyl amino acids.

18. The culture medium of claim 17, wherein the indanoyl amino acid is added to the culture medium in an amount effective to alter the ratio among taxanes produced by cells cultured in the culture medium relative to the ratio produced in the absence of the indanoyl amino acid.

19. The culture medium of claim 17, wherein the indanoyl amino acid is added to the culture medium in an amount effective to selectively increase baccatin III relative to the amount produced in the absence of the indanoyl amino acid.

20. The culture medium of claim 17, wherein the indanoyl amino acid is selected from the group consisting of 6-ethyl indanoyl isoleucine (6-EII), 6-Bromoindanoyl isoleucine (6-BII), and 1-oxo-indane-carboxy-(L)-isoleucine-methyl ester amide (1-OII).

21. The culture medium of claim 17, wherein the culture medium comprises at least one enhancement agent and/or inhibitor.

22. The culture medium of claim 17, wherein the culture medium comprises an auxin, a compound with auxin-like growth-regulator activity or both.

23. The culture medium of claim 17, wherein the culture medium comprises a silver ion, a silver compound, a silver complex or mixtures thereof.

24. The culture medium of claim 17, wherein the indanoyl amino acid is supplied in a protective amount with respect to silver toxicity.

25. The culture medium of claim 17, wherein the culture medium comprises an inhibitor of phenylpropanoid metabolism.

26. The culture medium of claim 25, wherein the inhibitor is a compound with a methylene-dioxy group.

27. The culture medium of claim 26, wherein the compound is 3,4-methylenedioxycinnamic acid, methylenedioxynitrocinnamic acid, methylenedioxyphenylpropionic acid, or 3,4-methylenedioxyphenylacetic acid.

28. The culture medium of claim 17, wherein the culture medium further comprises an amino acid.

29. The culture medium of claim 28, wherein the amino acid is glutamine.
